(19) United States
(12) Patent Application Publication
(10) Pub. No.: US 2015/0045856 A1
(43) Pub. Date: Feb. 12, 2015

(54) LICE EGG REMOVAL TOOL

(71) Applicant: MedEquity Capital LLC, Wellesley Hills, MA (US)

(72) Inventor: Wayne Brandon Ingersoll, Cambridge, MA (US)

(21) Appl. No.: 14/454,922

(22) Filed: Aug. 8, 2014

Related U.S. Application Data
(60) Provisional application No. 61/864,884, filed on Aug. 12, 2013.

(51) Int. Cl. A61N 1/32; A45D 24/30; A01M 1/22

(52) U.S. Cl. CPC A61N 1/322; A01M 1/223; A45D 24/30
USPC 607/79

(57) ABSTRACT

The present invention is relates to a tool for killing lice eggs comprising a body having proximal and distal ends and a comb comprising a first plurality of conductive tines alternating with a second plurality of conductive tines. A plurality of openings are defined by adjacent ones of the first and second plurality of conductive tines, wherein a width of the plurality of openings is less than or equal to 0.2 mm. A first electrical circuit is provided in electrical communication with the comb, the electrical circuit adapted to generate a potential difference between the first and second plurality of tines.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1 is a schematic illustration of a tool for killing lice eggs;

FIG. 2A is a picture of one embodiment of a comb for use with the tool of FIG. 1,

FIG. 2B is a schematic illustration of tines of the comb of FIG. 2A;

FIGS. 3A-3B are schematic illustrations of eggs positioned within hair on a scalp;

FIG. 4A is a schematic illustration of the tool of FIG. 1 in use; and

FIG. 4B is a schematic illustration of the tool of FIG. 1 in use, detailing the tool killing an egg.

### DETAILED DESCRIPTION

Lice are parasitic insects that live amongst hairs and feed upon small amounts of blood drawn from skin. For example, the head louse may be found amongst human hairs and feeds upon blood drawn from the scalp. Bites from lice may cause the scalp to become itchy and inflamed and persistent scratching may lead to skin irritation and possibly infection.

Lice reproduce by laying eggs. Lice eggs are positioned on hair shafts close to the scalp, where the temperature is suitable for keeping the eggs warm until they hatch. The eggs appear similar to dandruff; however, they cannot be removed by simply brushing or shaking the hair.

Although lice are not dangerous and do not spread disease, they are annoying and contagious. While many technologies and products exist to eliminate lice from hair, few effectively address the eggs those lice leave behind. Mature females lay between 3-4 eggs per day. If the lice are removed but not the eggs, those eggs will hatch within 8 days and reproduce to create a new infestation. Therefore it is desirable to remove the eggs before they hatch to avoid further infestation and transmission to others.

Accordingly, a continuing need exists for improved systems and methods for killing eggs.

Embodiments of the present disclosure present a tool for killing lice eggs and a corresponding method of use. In general, the tool includes a comb having a plurality of tines space by openings. A potential difference is also provided between adjacent tines. In use, the comb is run through the hair of a patient. When an egg bridges the opening between adjacent tines, a circuit is formed and current flows through the egg, between the adjacent tines. The current may kill the egg outright by electrocution or induce sufficient damage (e.g., cell wall damage) such that the egg dies some time later (e.g., due to dehydration from loss of water through the cell wall damage). The tool is adapted to be positioned very close to a patient's skin, because the highest concentration of eggs is located within ¼″ from the scalp. This provides a higher likelihood of engaging eggs when the comb of the tool is run through the patient's hair. The openings between the tines are also selected to be less than the average width of a egg (which oblong dimensions are on average are 0.3×0.8 mm) , ensuring that eggs engaged by the tines are captured and electrocuted. So configured, embodiments of the tool significantly provide significantly improved ability to capture and kill eggs.

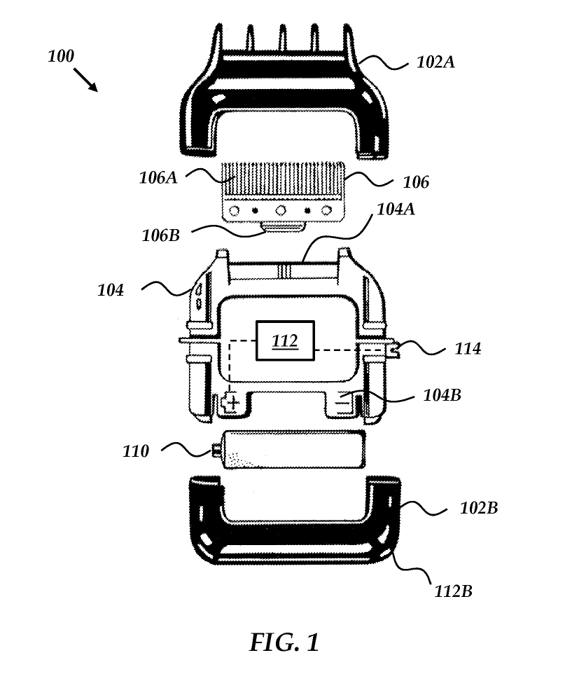

An exploded view of an embodiment of a tool 100 for killing eggs is illustrated in FIG. 1. The tool includes an outer housing having a distal portion 102A and a proximal portion 102B (collectively outer housing 102). In certain embodiments, the outer housing 102 may be formed from plastic (e.g., an injection molded part). The tool 100 may further include an inner housing 104, a comb 106, and a power source 110. The proximal portion of the outer housing 102B may be adapted to receive the power source 110 and a proximal portion of the inner housing 104B. The distal portion of the outer housing 102B may be adapted to receive the comb 106 and the distal portion of the inner housing 104B. So assembled, the tool 100 may be dimensioned for grasping in a user's hand.

The inner housing 104 may include sockets 104A and 104B at distal and proximal ends, respectively in electrical communication with a plurality of circuits 112. In an embodiment, the proximal socket 104E may be adapted to receive the power source 110 and place the power source 110 in electrical communication with the plurality of circuits 112. The distal socket 104A may be adapted to receive a portion of the comb 106 and place the comb in electrical communication with the plurality of circuits 112 and the power supply 110. As discussed in greater detail below, the plurality of electrical circuits 112 may be adapted to control a voltage applied to the comb 106.

In an embodiment, the power source 110 may be a primary or secondary battery (e.g., a 1.5V battery). The power source 110 may be integrated with the tool 100 or adapted for removal. In alternative embodiments, the power source 110 may be an adaptor for electrically communicating with a remote power source (e.g., a connector for receiving electrical power from a wall socket).

The comb 106 may include tines 106A positioned at a distal end and a connector 106B positioned at a proximal end. The connector may be adapted for reception by the distal socket of the inner housing 104B and place the tines 106A in electrical communication with the power source 110 and the plurality of circuits 112.

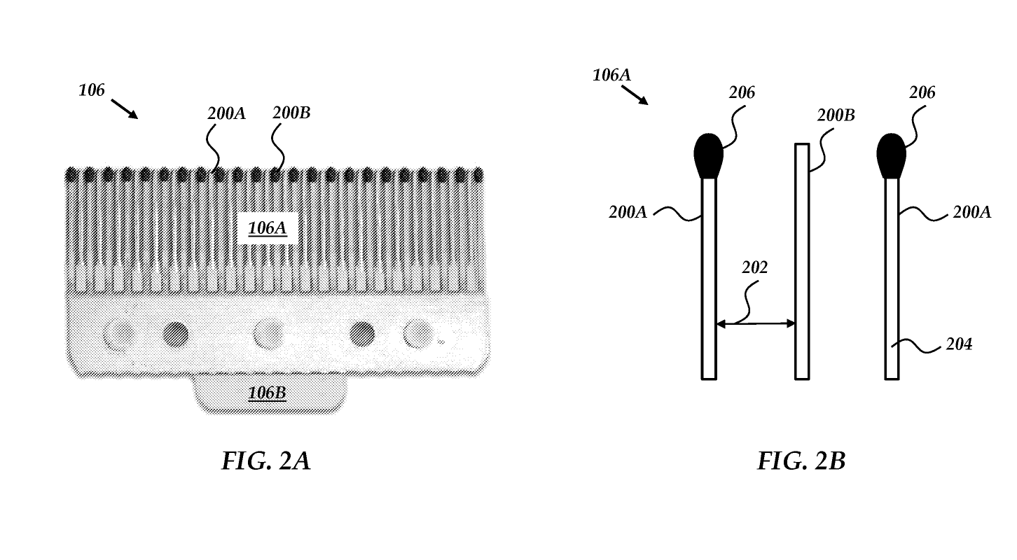

With further reference to FIG. 2, the tines 106A may include a first plurality of tines 200A and a second plurality of tines 200B. The second plurality of tines 200B may be interposed between the first plurality of tines 200A. So configured, the tines 106A are composed of alternating members of the first and second plurality of tines 200A, 200B, separated by openings 202.

The tines 106A may further be formed from an electrically conductive material. Examples may include, but are not limited to, metals, conductive polymers, etc. The plurality of electrical circuits 112 and the power supply may be in electrical communication with the tines 106A and provide a potential difference between the first plurality of tines 200A and the second plurality of tines 200B.

In an embodiment, the plurality of electrical circuits 112 may control the magnitude of the potential difference between the first and second plurality of tines 200A, 200B. For example, the plurality of electrical circuits 112 may be adapted to provide a potential difference between the first and second plurality of tines 200A, 200B which is significantly greater than the voltage of the power supply 110. In one embodiment, the power supply 110 may possess a voltage of 1.5V and the plurality of electrical circuits 112 may provide a potential difference between the first and second plurality of tines 200A, 200B within the range between about 100V to about 300V, more preferably about 250V to about 300V with low amps (e.g., approximately 1 A). In a further embodiment, the plurality of electrical circuits 112 may be in communication with a user-selectable on-off switch 114 for turning on and off the potential difference between the first and second plurality of tines 200A, 200B.

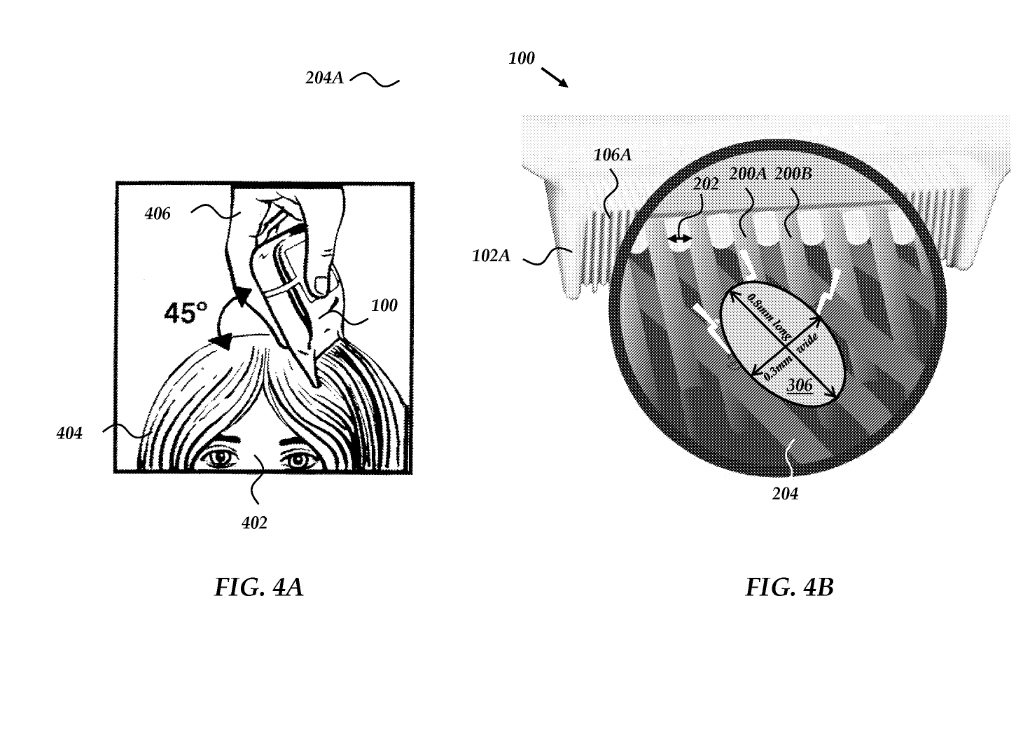

FIGS. 4A-4B illustrate use of the tool 100 with hair 404 of a patient 402. While hair from a patient's scalp is illustrated in FIGS. 4A-4B, it may be understood that embodiments of the tool 100 may be employed with any hair infested by eggs. An operator 406 engages the comb 106 with the hair 404. The operator 406 may be the patient 402 or another individual.

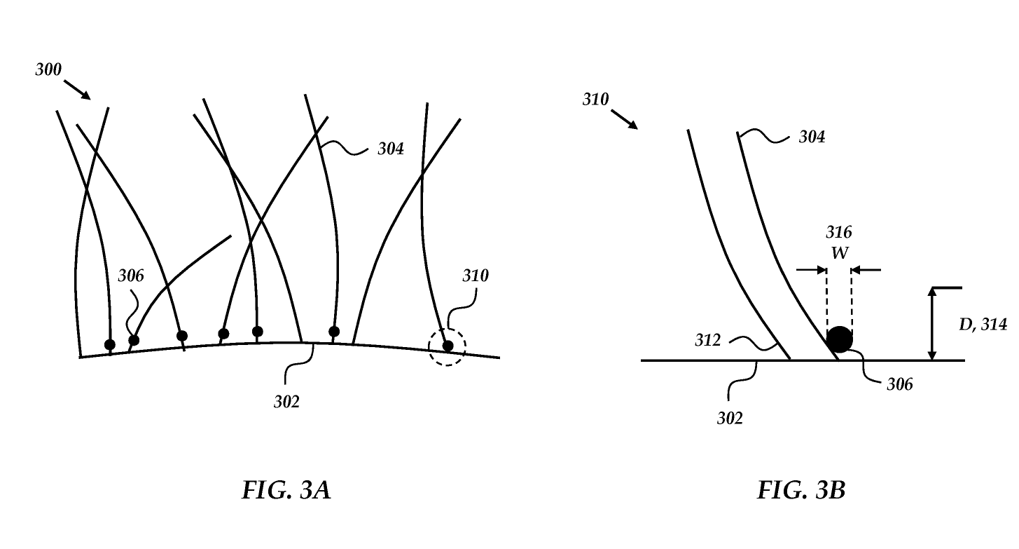

Embodiments of the tool 100 arc specifically designed to safely and effectively capture eggs. For example, with reference to FIGS. 3A-3B, skin 300 is illustrated with hair shafts 302 extending outward from the skin surface 304. Lice eggs 306 are laid very close to the root of a hair shaft 302 and attached to the shaft 302 at or very near the skin surface 302. Unless a concerted effort is made to keep the tines 106A on or near the user's skin so the tines 106A will consistently sweep within approximately ¼″ or less of the scalp, the tines will miss the eggs. Missed eggs will later hatch and continue the cycle of infestation. Accordingly, embodiments of the tool 100 are adapted so that the tines 106A may be positioned by less than a distance D, 314 from the user's skin (e.g., less than ¼ inch).

For example, with further reference to FIG. 2B, the first plurality of tines 200A includes an elongate shaft 204 and an insulating tip 206 at a distal end. The insulating tip 206 may be ensure that the first plurality of tines 200A do not form a short circuit with the second plurality of tines 200B if the distal ends of the first and second plurality of tines 200A, 200B contact each other. The first plurality of tines 200A may also be formed with a shaft 204 that is longer than that of the second plurality of tines 200B so that the distal ends of the first plurality of tines 200A extend further outward from the comb than the second plurality of tines 200B. So configured, contact of the comb 106 with a user's skin (e.g., scalp) will place the insulating tips of the first plurality of tines 200A into contact with the user's skin, avoiding possible electrical shock to the user when the potential difference between the first and second plurality of tines 200A, 200B is present.

In further embodiments, the openings 202 between the tines 106A may be adapted to ensure capture of at least a portion of the eggs impinging the openings 202. For example, as illustrated in FIGS. 3B, 4B, lice eggs are approximately 0.3 mm in width and approximately 0.8 mm in length, on average (see, e.g., width W, 316). Thus, the tine spacing 202 may be selected to be less than this average width so that a patient's hair may pass through the openings 202 but eggs (e.g., eggs 306) are inhibited from passing through the openings 202 (see, e.g., FIG. 4B). For example, the tine spacing 202 may be less than or equal to 0.2 mm so as to ensure the eggs span the width of the tines necessary to conduct an electrical charge.

In an embodiment, the tool 100 may be used as follows. The on/off switch 114 may be activated to generate a potential difference between the first and second plurality of tines 200A, 200B. The patient's hair 404 may be divided into sections, each of which is combed by passing the hair 404 through the openings 202 in the tines 106. In certain embodiments, the tines 106 are positioned approximately ¼ inch or less from the patient's skin (e.g., scalp) during combing. The comb 106 may also be oriented at an angle of approximately 45° with respect to the roots of the hair 404. These operations may be repeated on all hair. In certain embodiments, the combing process may be performed for a minimum of five minutes, preferably at least ten minutes, more preferably, at least fifteen minutes to adequately ensure that the comb 106 engages all the patient's hair 404.

In further embodiments, the tool 100 may be used at least once daily for consecutive days. It may be understood that, when used, the tool 100 may kill both live lice and eggs. However, it is not guaranteed that a single use of the tool 100 may capture and kill every louse and egg infesting a patient's hair. An egg hatches approximately eight days from the time it is laid by a live louse. Thus, failure to remove all live lice and eggs from a patient's hair will result in re-infestation. Accordingly, by using the tool 100 on multiple, consecutive days, the likelihood of removing all lice and eggs from the patient's hair is increased after each use. In an embodiment, the tool 100 may be used one each day for at least eight consecutive days, preferably at least ten consecutive days.

Embodiments of the tool 100 may be further employed for preventative use. As discussed above, a use duration of at least approximately five minutes is sufficient to cover an entire head of hair, on average. The time for a new egg to hatch into a live louse is approximately eight days. Accordingly, use of the tool 100 once daily for at least eight consecutive days, as a preventative measure, may ensure that any new lice are killed before they can have the opportunity to reproduce in significant numbers. If a new louse is detected using the tool 100 for at least about five minutes per week, then a more thorough daily combing regimen, as discussed above, can then be used to eliminate further lice or eggs and prevent future infestation.

The tool 100 may also make an audible signal when lice or eggs are detected. For example, the plurality of circuits may be in further communication with a sound generator 120 (see, e.g., FIG. 1). The sound generator 120 may be adapted to produce an audible noise when a potential difference is produced between the tines 106A and an egg (or louse) is not present to bridge one of the openings 202. The sound generator 120 may be further adapted to not produce an audible noise when an egg (or louse) is present to bridge one of the openings 202 and current flows between the tines 106A bridged by the egg. As a result, the patient and/or operator of the tool 100 may be alerted to the presence of new eggs or lice and perform additional combing to further eliminate lice and eggs and to prevent further infestation.

The terms comprise, include, and/or plural forms of each are open ended and include the listed parts and can include additional parts that are not listed. The term and/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

What is claimed is:

1. A tool for killing lice eggs, comprising:
a body having proximal and distal ends;
a comb comprising a first plurality of conductive tines alternating with a second plurality of conductive tines;
a plurality of openings defined by adjacent ones of the first and second plurality of conductive tines, wherein a width of the plurality of openings is less than or equal to 0.2 mm;
a first electrical circuit in electrical communication with the comb, the electrical circuit adapted to generate a potential difference between the first and second plurality of tines.

2. The tool of claim 1, further comprising a second electrical circuit formed by a lice egg in contact with adjacent tines of the first and second plurality of tines.

3. The tool of claim 2, wherein a current flowing within the second electrical circuit is driven by the potential difference and possesses a magnitude sufficient to electrocute the lice egg.

4. The tool of claim 1, wherein the potential difference is greater than or equal to 100V.

5. The tool of claim 1, wherein each of the first plurality of conductive tines further comprises an insulating tip positioned at its respective distal end.

6. The tool of claim 1, further comprising a noise generator in electrical communication with the first electrical circuit, wherein the noise generator is adapted to produce an audible signal when respective ones of the plurality of openings are not bridged by a lice egg and wherein the noise generator is adapted to not produce an audible signal when one or more of the plurality of openings is bridged by a lice egg

7. A method of killing lice eggs, comprising:
positioning a comb from a skin surface from which a plurality of hair shafts extend, the hair shafts including a plurality of lice eggs attached thereto, wherein the comb is positioned at a distance less than or equal to 0.25 inches;
wherein the comb comprises a first plurality of conductive tines alternating with a second plurality of conductive tines and wherein the first and second plurality of tines define a plurality of openings there-between, the plurality of openings each having a width less than or equal to 0.2 mm;
generating a potential difference between the first and second plurality of tines;
moving the comb with respect to the skin surface so as to urge at least a portion of the plurality of hair shafts to pass through the plurality of openings, wherein the distal end of the tines are maintained at a distance less than or equal to 0.25 inches from a skin surface during said moving;
forming an electrical circuit by a lice egg in contact with adjacent tines of the first and second plurality of tines; and
driving a current within the electrical circuit by the potential difference, the current possessing a magnitude sufficient to electrocute the lice egg.

8. The method of claim 7, further comprising repeating the moving of the comb for a cumulated total of 5 minutes.

9. The method of claim 8, further comprising performing the method at least one time daily for at least eight consecutive days.

10. The method of claim 7, wherein positioning the comb further comprises orienting the comb such that the first and second plurality of tines are positioned an angle of approximately 45 degrees with the skin surface
